(19) United States
(12) Patent Application Publication
(10) Pub. No.: US 2011/0152686 A1
(43) Pub. Date: Jun. 23, 2011

(54) ULTRASOUND IMAGING

(75) Inventors: Allen Snook, Snohomish, WA (US); Rohit Garg, Kirkland, WA (US); Michael Vion, La Chaussee St Victor (FR)

(73) Assignee: KONINKLIJKE PHILIPS ELECTRONICS N.V., Eindhoven (NL)

(21) Appl. No.: 13/060,285

(22) PCT Filed: Aug. 24, 2009
(86) PCT No.: PCT/IB2009/053717

§ 371 Date: Feb. 23, 2011

Related U.S. Application Data
(60) Provisional application No. 61/093,744, filed on Sep. 3, 2008.

(51) Int. Cl. A61B 8/14

(52) U.S. Cl. 600/443

(57) ABSTRACT

An ultrasound imaging system (UIS) comprises a user interface (UIF) that allows an operator to request visual aid. In response to a request for visual aid, a controller (CTRL) retrieves from a database a visual representation of a preferred manner of applying a probe to a patient, and causes a display device (DPL) to render the visual representation. Preferably, but not necessarily, the controller (CTRL) detects a step of a workflow protocol program (WPP) that is being carried out. The controller (CTRL) then selects from the database a visual representation pertaining to the step that is being carried out.

### FIELD OF THE INVENTION

An aspect of the invention relates to an ultrasound imaging system. The ultrasound imaging system may be in the form of, for example, a portable device that allows telemedicine applications. Other aspects of the invention relate to a method of ultrasound imaging, and a computer program product.

### BACKGROUND OF THE INVENTION

In medical applications, an ultrasound imaging system is typically used for acquiring ultrasound scan data relating to a particular body portion of a patient. The ultrasound scan data is visually rendered on a display device as an image, or a sequence of images, for carrying out a diagnosis, which will serve as a basis for medical treatment.

The international application published under number WO 2006/038181 describes an ultrasonic diagnostic system that contains one or more exam protocols, which guide an operator in carrying out one or more standardized ultrasound exams. During the conduct of an exam protocol template protocol steps are available. The operator can augment the standard protocol by selecting one of these template protocol steps and giving it a unique name for the current protocol. The execution of this added step will automatically invoke the diagnostic tools, such as measurements and calculations, which are inherent in the cloned conventional protocol step. In addition, the operator can add other diagnostic tools to the added step. The results of the standard and customized protocol steps are automatically recorded in the proper sequence and context of an automatically produced diagnostic report.

An operator of an ultrasound imaging system needs to have a considerable level of skill and experience in order to acquire ultrasound scan data that has sufficient diagnostic value. Ultrasound imaging is a specialist's job, which involves placing and moving an ultrasound probe in a particular fashion in order to obtain ultrasound scan data of sufficient diagnostic value. A protocol driven workflow as described in the aforementioned international application gives little guidance on these aspects. In many cases, there is no need for such guidance because only skilled, experienced persons are allowed to operate an ultrasound imaging system for the purpose of carrying out a diagnosis.

However, there may be locations or situations where there is a need to acquire ultrasound scan data of sufficient diagnostic value, but where no imaging specialists are available. Examples include rural clinics, first responder interventions, less-developed countries, and battlefields. In these cases, ultrasound imaging generally needs to be performed by less experienced operators, who may not be familiar with best practices for scanning a given anatomy or disease profile. This could result in misdiagnosis. Even if an operator has had some basic training, he or she may need to operate in a stressful situation and may therefore find it difficult to recall their training. The operator may need to carry out a relatively great number of scans before arriving at a scan that provides adequate diagnostic information. This implies delay of treatment. In serious situations, all this may lead to the loss of the patient.

### SUMMARY OF THE INVENTION

There is a need for an improved ultrasound imaging system, which allows a less experienced operator to quickly acquire ultrasound images that have sufficient diagnostic value.

In accordance with an aspect of the invention, an ultrasound imaging system comprises a user interface that allows an operator to request visual aid. In response to a request for visual aid, a controller retrieves from a database a visual representation of a preferred manner of applying a probe to a patient, and causes a display device to render the visual representation.

For example, a less experienced operator may mimic a probe placement and a scanning motion that is displayed on the display device as a preferred manner of applying a probe to a patient. This may significantly increase the chance of capturing ultrasound scan data of sufficient diagnostic value.

An implementation of the invention advantageously comprises one or more of the following additional features, which are described in separate paragraphs that correspond with individual dependent claims.

Preferably, the controller detects a step of a workflow protocol program that is being carried out, and selects from the database a visual representation pertaining to the step that is being carried out.

The ultrasound imaging system preferably comprises a storage module with at least one visual representation of a preferred manner of acquiring ultrasound image.

The ultrasound imaging system preferably comprises a communication module for transmitting acquired ultrasound scan data to a remote location.

The database may comprise at least one still picture representing a preferred manner of applying a probe to a patient.

The database may comprise at least one video representing a preferred manner of applying a probe to a patient.

The database preferably comprises audio information in association with at least one visual representation of a preferred manner of applying a probe to a patient.

The audio information preferably comprises verbal instructions for applying a probe to a patient.

A detailed description, with reference to drawings, illustrates the invention summarized hereinbefore as well as the additional features.

### BRIEF DESCRIPTION OF THE DRAWINGS

FIG. 1 is a block diagram that illustrates an ultrasound imaging system.

FIG. 2 is a flow chart diagram that illustrates a series of steps that the ultrasound imaging system carries out.

### DETAILED DESCRIPTION OF THE INVENTION

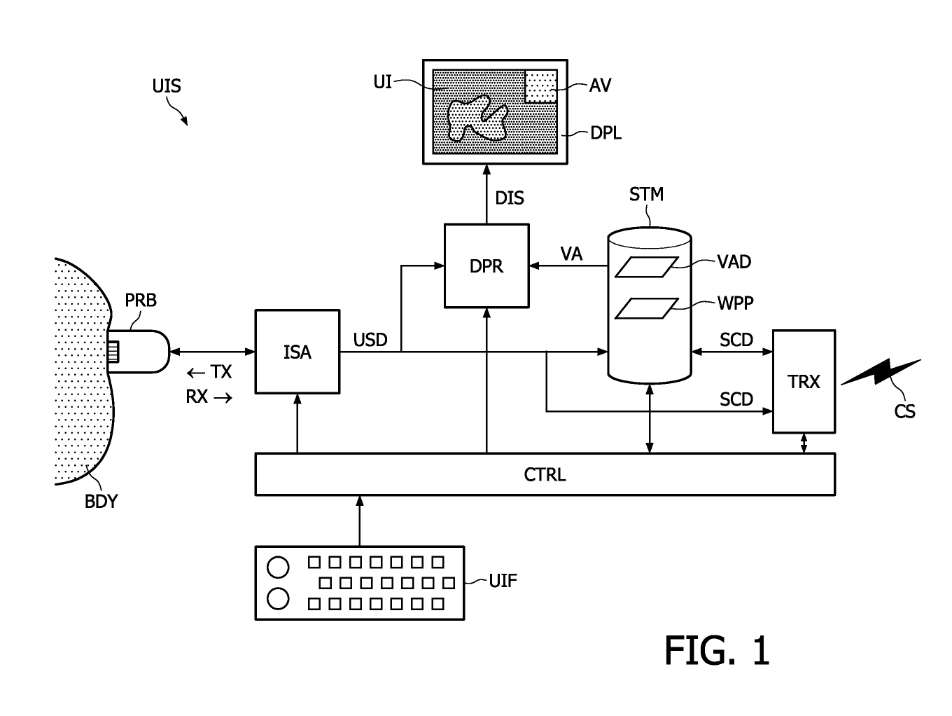

FIG. 1 illustrates an ultrasound imaging system UIS, which may be in the form of a portable device. The ultrasound imaging system UIS comprises a probe PRB, an ultrasound scanning assembly ISA, a display processor DPR, a display device DPL, a controller CTRL, and a user interface UIF. The probe PRB may comprise, for example, a two-dimensional array of piezoelectric transducers. The ultrasound scanning assembly ISA may comprise an ultrasound transmitter and an ultrasound receiver, which may each include a beam-forming module. The ultrasound scanning assembly ISA may further comprise one or more filter modules and a so-called B-mode processing module.

The controller CTRL may be in the form of, for example, a suitably programmed processor. The user interface UIF may comprise physical elements, such as, for example, various alphanumerical keys, knobs, and a mouse or trackball. However, the user interface UIF may also comprise software components, which the controller CTRL carries out. For example, a software component may cause the display device DPL to display a menu from which an operator may select an item by pressing a particular key or by moving a cursor to the item as displayed.

The ultrasound imaging system UIS further comprises the following functional entities: a storage module STM and a communication module TRX. The storage module STM may be in the form of, for example, a hard disk. The storage module STM comprises a workflow protocol program WPP, which the controller CTRL may execute, and, in addition, an audiovisual aid database VAD. The communication module TRX may comprise, for example, a transceiver circuitry for wireless communication or for communication via a wired network. The communication module TRX may further comprise one or more interfaces that implement one or more communication protocols such as, for example, protocols used for communication via the Internet. The communication module TRX allows the ultrasound imaging system UIS to transfer acquired ultrasound images to remote locations.

The ultrasound imaging system UIS basically operates as follows. It is assumed that an operator has just switched on the ultrasound imaging system UIS or has just initiated an ultrasound image acquisition process. In response, the controller CTRL executes the workflow protocol program WPP, which is stored in the storage module STM. The workflow protocol program WPP provides a structured sequence of steps, which are needed for acquiring ultrasound images in a particular mode. In a particular step, the workflow protocol program WPP may invite the operator to specify or set one or more parameters through the user interface UIF.

In a scanning step, the probe PRB is in contact with a body portion BDY of a patient as illustrated in FIG. 1. The ultrasound scanning assembly ISA applies a set of transmission signals TX to the probe PRB and receives a set of reception signals RX from the probe PRB. The set of reception signals RX comprises reflections of the transmission signals TX. These reflections occur within the body portion BDY of the patient. The set of reception signals RX are processed so as to obtain ultrasound scan data USD. The ultrasound scan data USD may be in the form of, for example, so-called B-mode images. The ultrasound scan data USD may provide a two-dimensional or a three-dimensional representation. In the latter case, the ultrasound scan data USD may be designated as volume data.

The display processor DPR generates display images DIS that typically comprise a visual representation the ultrasound scan data USD, which the ultrasound scanning assembly ISA provides. The display device DPL displays the display images DIS. The display images DIS may comprise one or more auxiliary visual representations, which need not necessarily be based on the ultrasound scan data USD. An auxiliary visual representation may be retrieved from the storage module STM, as will be described in greater detail hereinafter. FIG. 1 illustrates an example in which a display image comprises an ultrasound image UI, which is the visual representation of the ultrasound scan data USD, and a sub-image AV that comprises an auxiliary visual representation.

The ultrasound scan data USD and the visual representation thereof have a diagnostic value that largely depends on placement of the probe PRB and scanning motion, if any. In this respect, it should be noted that different anatomies typically require different probe placements and different scanning motions. The diagnostic value of the ultrasound scan data USD will therefore largely depend on the operator's ability to appropriately place the probe PRB and appropriately move the probe PRB for the anatomy of interest. A lack of experience may prevent the operator from acquiring ultrasound scan data USD of sufficient quality from a diagnostic point of view. A situation of duress may also prevent the operator from acquiring ultrasound scan data USD that allows a reliable diagnosis.

The ultrasound imaging system UIS illustrated in FIG. 1 is arranged to enhance the operator's ability to acquire ultrasound scan data USD of sufficient quality. To that end, the user interface UIF may be provided with a help key, which the operator may depress in order to obtain visual aid. The user interface UIF may also display a help icon on the display device DPL. The help icon may be overlaid on an image pertaining to the workflow protocol program WPP or an ultrasound image. In any case, the user interface UIF allows the operator to indicate that he or she requires visual aid.

In response to a request for visual aid, the controller CTRL selects visual aid data VA from the audiovisual aid database VAD and applies the visual aid data VA to the display processor DPR as an auxiliary visual representation. The sub-image AV illustrated in FIG. 1 may thus display the visual aid data VA that has been selected from the audiovisual aid database VAD. Alternatively, the visual aid data VA may be displayed in a full screen mode. In case the visual aid data VA is rendered in the form of a sub-image, the ultrasound imaging system UIS allows the operator to move the sub-image and to resize the sub-image. To that end, the controller CTRL may comprise one or more specific software components that interact with physical elements of the user interface UIF, such as, for example, a mouse and one or more buttons or keys.

The visual aid data VA may comprise one or more still pictures, or one or more videos, or a combination of both. Multiple still pictures or multiple videos may illustrate alternative ultrasound imaging techniques. This may be particularly useful in cases where, for example, it is difficult to apply ultrasound imaging to a patient, or where an injury prevents a primary mode of ultrasound imaging. The visual aid data VA may also comprise sound and verbal instructions, which may be rendered by means of a loudspeaker that forms part of the ultrasound imaging system UIS.

The visual aid data VA, which is displayed, illustrates a best practice in probe placement and scanning motion for the anatomy of interest. The anatomy of interest to which the visual aid data VA pertains, may have been identified in one or more steps that the workflow protocol program WPP has carried out. The operator may mimic the probe placement and the scanning motion that is displayed on the display device DPL in the sub-image AV, or in a full screen mode, or otherwise. This may significantly increase the chance of capturing ultrasound scan data USD of sufficient diagnostic value. The ultrasound imaging system UIS preferably allows the operator to rewind, loop, fast forward, play at a lower speed, and pause a video comprised in the visual aid data VA.

Accordingly, even in case the operator lacks experience, he or she may be able to operate the ultrasound imaging system UIS in such a fashion that the ultrasound scan data USD has sufficient diagnostic value. However, the operator may not be able to carry out a diagnosis on the basis of the display images DIS that represent the ultrasound scan data USD. What is more, there may not be any person capable of carrying out a diagnosis in the vicinity of the ultrasound imaging system UIS. This may be the case in, for example, rural clinics, less-developed countries, and battlefields. In such cases, the ultrasound scan data USD, or the display images DIS generated therefrom, or both, should be transferred to a remote location where a specialist resides capable of carrying out a diagnosis. This can be qualified as telemedicine.

The ultrasound imaging system UIS illustrated in FIG. 1 allows the operator, or any other person for that matter, to transmit the ultrasound scan data USD, or the images generated therefrom, or both, to a remote specialist. The operator may initiate a data transmission while acquiring the ultrasound scan data USD, or after having acquired the ultrasound scan data USD. In the latter case, the ultrasound scan data USD may be stored in, for example, the storage module STM. The controller CTRL may control the data transmission on the basis of transmission parameters that have been specified by the operator, or another person. The controller CTRL causes the ultrasound scan data USD to be applied to the communication module TRX. The controller CTRL may further appropriately configure the communication module TRX so that the ultrasound scan data USD is transmitted in accordance with the transmission parameters that have been specified. For example, in case the data transmission takes place via the Internet, the operator may specify a URL, or an e-mail address, under which the remote specialist can be reached. In case the data transmission takes place via a wireless radio, the operator may specify a particular channel. Encryption may be used for transmitting the ultrasound scan data USD.

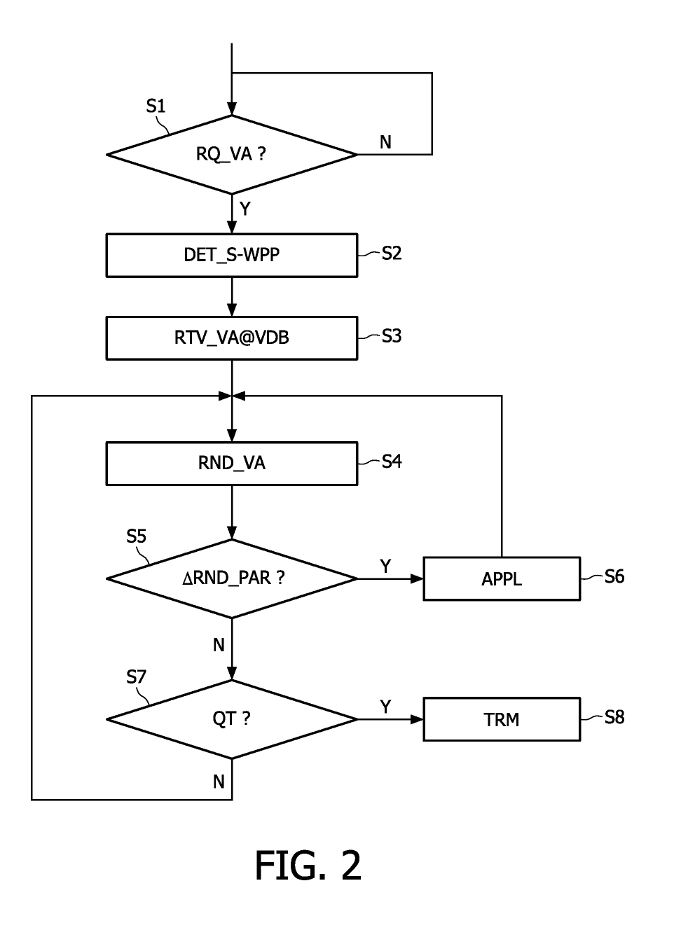

FIG. 2 illustrates a series of steps S1-S8 that the controller CTRL illustrated in FIG. 2 may carry out in order to provide visual aid data VA on request. The series of steps S1-S8 may form part of, for example, the workflow protocol program WPP that is stored in the storage module STM. FIG. 2 may be regarded as a flowchart representation of a software program, that is, a set of instructions, which causes the controller CTRL to carry out various operations described hereinafter with reference to FIG. 2.

In step S1 (RQ_VA ?), the controller CTRL checks whether there is a request for visual aid, or not. As mentioned hereinbefore, an operator may express a request for visual aid by means of the user interface UIF. That is, the controller CTRL may continuously monitor the user interface UIF, as it were, to determine whether a request for visual aid has been expressed, or not. In case there is a request for visual aid, the controller CTRL subsequently carries out step S2.

In step S2 (DET_S-WPP), the controller CTRL detects the step of the workflow protocol program WPP that is currently being carried out. That is, the controller CTRL obtains contextual information concerning the ultrasound imaging acquisition process, as well as a point that has been reached in this process when the request for visual aid was made.

In step S3 (RTV_VA@VDB), the controller CTRL retrieves visual aid data VA from the audiovisual aid database VAD that pertains to the current step of the workflow protocol program WPP. More specifically, the visual aid data VA that has been retrieved is applied to the display processor DPR. The audiovisual aid database VAD may comprise various elements pertaining to various steps in the workflow protocol program WPP. Nonetheless, an element may pertain to various different steps; different steps may share a particular element. The controller CTRL selects one or more elements that are relevant for the current step of the workflow protocol program WPP.

In step S4 (RND_VA), the controller CTRL controls a rendering of the visual aid data VA in accordance with applicable rendering parameters. Initially, the visual aid data VA may be rendered in a default fashion, similar to what happens if a play button of a media player is depressed.

In step S5 (ΔRND_PAR?), the controller CTRL checks whether the operator has specified a rendering parameter that is different from the current one. The operator may specify various different rendering-related actions, such as, for example, play, rewind, loop, fast forward, play at slower speed, and pause. Other rendering-related actions, which may be typically used for video, are possible. In case the visual aid data VA comprises audio, the operator may mute this audio. This feature may be useful in case, for example, the ultrasound scan data USD comprises information that can be rendered as audio. Accordingly, the operator can decide to listen, as it were, to a biological process within the body portion BDY of the patient, rather than to verbal instructions associated with the visual aid data VA.

In step S6 (APPL), the controller CTRL applies one or more rendering parameters that the operator has specified in step S5. The controller CTRL continues rendering the visual aid data VA in accordance with the applicable rendering parameters, which is symbolized by means of a return to step S4. In case no different rendering parameters have been specified, the controller CTRL carries out step S7 subsequent to step S5.

In step S7 (QT?), the controller CTRL checks whether the operator has made a request to stop rendering the visual aid data VA. In case the operator has made such a request, the controller CTRL causes the rendering to stop in step S8 (TRM). This may involve, for example, closing the sub-image AV illustrated in FIG. 1. In case the operator has not made such a request, the controller CTRL continues the rendering of the visual aid data VA in accordance with the applicable parameters. FIG. 2 symbolizes the same by means of a return to step S4.

In case the workflow protocol program WPP reaches a new step, while the operator has not requested the rendering to be stopped, the controller CTRL may carry out steps S2-S8 for the new step in the workflow protocol program WPP. In such a case, the operator continues to receive visual aid throughout several steps of the workflow protocol program WPP.

### Concluding Remarks:

The detailed description hereinbefore with reference to the drawings is merely an illustration of the invention and the additional features, which are defined in the claims. The invention can be implemented in numerous different ways. In order to illustrate this, some alternatives are briefly indicated.

The invention may be applied to advantage in numerous types of products or methods related to ultrasound imaging. For example, the invention may be applied in a portable computer, which is configured for ultrasound imaging purposes. The portable computer may interface with, for example, a dedicated ultrasound imaging module that comprises, for example, one or more beamformers as well as other circuits for applying activation signals to a probe and for processing reception signals from the probe. Such a dedicated ultrasound imaging module will typically comprise analog to digital converters and digital to analog converters.

A visual representation of a preferred manner of applying a probe to a patient, need not necessarily concern a probe with ultrasound transducers. The probe may be, for example, an ECG probe (ECG is an acronym for electrocardiogram). An operator may mimic a preferred manner of ECG lead placement that is rendered on the display device.

There are numerous ways in which an ultrasound imaging system in accordance with the invention may retrieve a visual representation, which provides visual aid, from a database. FIG. 1 illustrates an example in which the database or at least a portion thereof, resides in a storage module that forms part of the ultrasound imaging system. In another implementation, the database may reside in a remote storage module, which can be accessed by means of, for example, a communication module. Referring to FIG. 1, the controller CTRL may be programmed so that a link with a remote database is established from which the visual representation of interest is retrieved.

In broad terms, there are numerous ways of implementing functional entities by means of hardware or software, or a combination of both. Although software-based implementations as indicated in the detailed description are generally preferred, hardware-based implementations are by no means excluded. For example, any functional entity described hereinbefore may equally be implemented by means of a dedicated circuit, which has a particular topology defining one or more operations that the functional entity concerned carries out. Hybrid implementations are also possible in the sense that a system, or a functional entity comprises therein, comprises one or more dedicated circuits as well as one or more suitably programmed processors.

Although a drawing shows different functional entities as different blocks, this by no means excludes implementations in which a single entity carries out several functions, or in which several entities carry out a single function. In this respect, the drawings are very diagrammatic. For example, referring to FIG. 1, a single programmable circuit may be programmed to carry out operations belonging to the controller CTRL as well as operations belonging to the ultrasound scanning assembly ISA.

There are numerous ways of storing and distributing a set of instructions, that is, software, which allows an ultrasound imaging system to operate in accordance with the invention. For example, software may be stored in a suitable medium, such as an optical disk or a memory circuit. A medium in which software stored may be supplied as an individual product or together with another product, which may execute software. Such a medium may also be part of a product that enables software to be executed. Software may also be distributed via communication networks, which may be wired, wireless, or hybrid. For example, software may be distributed via the Internet. Software may be made available for download by means of a server. Downloading may be subject to a payment.

The remarks made herein before demonstrate that the detailed description with reference to the drawings, illustrate rather than limit the invention. There are numerous alternatives, which fall within the scope of the appended claims. Any reference sign in a claim should not be construed as limiting the claim. The word “comprising” does not exclude the presence of other elements or steps than those listed in a claim. The word “a” or “an” preceding an element or step does not exclude the presence of a plurality of such elements or steps. The mere fact that respective dependent claims define respective additional features, does not exclude a combination of additional features, which corresponds to a combination of dependent claims.

1. An ultrasound imaging system comprising:
a user interface allowing an operator to request visual aid; and
a controller arranged to detect a step of a workflow protocol program that is being carried out, and to select from a database, in response to a request for visual aid, a video pertaining to the step that is being carried out and representing a preferred manner of applying a probe to a patient, and to cause a display device to render the video.

2. (canceled)

3. An ultrasound imaging system according to claim 1, comprising a storage module with at least one video representing a preferred manner of acquiring ultrasound image.

4. An ultrasound imaging system according to claim 1, comprising a communication module for transmitting acquired ultrasound scan data to a remote location.

5. An ultrasound imaging system according to claim 1, the database comprising, in conjunction with the video, at least one still picture representing a preferred manner of applying a probe to a patient.

6. (canceled)

7. An ultrasound imaging system according to claim 1, the database comprising audio information in association with the video.

8. An ultrasound imaging system according to claim 7, the audio information comprising verbal instructions for applying a probe to a patient.

9. A method of ultrasound imaging involving an ultrasound imaging system comprising a user interface allowing an operator to request visual aid, the method comprising:
a detection step in which a step of a workflow protocol program that is being carried out is detected; and
a video provision step, which is carried out in response to a request for visual aid, in which step a video pertaining to the step of workflow protocol program that is being carried out is selected from a database, the video representing a preferred manner of applying a probe to a patient, and in which a display device is caused to render the video.

10. A computer program product for an ultrasound imaging system comprising a user interface allowing an operator to request visual aid a programmable processor,
the computer program product comprising a set of instructions, which when loaded into the programmable processor, enables the programmable processor to carry out the method according to claim 7.
